(19) United States
(12) Patent Application Publication
(10) Pub. No.: US 2008/0058558 A1
(43) Pub. Date: Mar. 6, 2008

(54) PREPARATION OF DECABROMODIPHENYL OXIDE

(75) Inventors: Arthur G. Mack, Prairieville, LA (US); Paul F. Ranken, Baton Rouge, LA (US); Robert E. Williams, Magnolia, AR (US)

Correspondence Address: ALBEMARLE CORPORATION, 451 FLORIDA STREET, BATON ROUGE, LA, 70801-1765, US

(73) Assignee: ALBEMARLE CORPORATION, Baton Rouge, LA (US)

(21) Appl. No.: 11/843,750

(22) Filed: Aug. 23, 2007

Related U.S. Application Data
(60) Provisional application No. 60/823,827, filed on Aug. 29, 2006.

(51) Int. Cl. C07C 41/22

(52) U.S. Cl. 568/639

(57) ABSTRACT

This invention provides a process for producing decabromodiphenyl oxide from a liquid mixture. The liquid mixture is derived from bromine, a Lewis acid catalyst, and a diphenyl oxide species selected from the group consisting of
(i) diphenyl oxide,
(ii) partially brominated diphenyl oxide,
(iii) decabromodiphenyl oxide having about 0.5% or more nonabromodiphenyl oxide, and
(iv) any combination of (i)-(iii).
   The process comprises distilling bromine and hydrogen bromide from the liquid mixture while feeding bromine to the liquid mixture.

### REFERENCE TO RELATED APPLICATION

This application claims the benefit and priority of U.S. Provisional Application No. 60/823,827, filed Aug. 29, 2006, the disclosure of which is incorporated herein by reference.

### TECHNICAL FIELD

This invention relates to the preparation of high purity decabromodiphenyl oxide products.

### BACKGROUND

Decabromodiphenyl oxide (DBDPO) is a time-proven flame retardant for use in many flammable macromolecular materials, e.g., thermoplastics, thermosets, cellulosic materials, and back coating applications.

DBDPO is presently sold as a powder derived from the bromination of diphenyl oxide or a partially brominated diphenyl oxide containing an average of about 0.7 bromine atom per molecule of diphenyl oxide. Such bromination is conducted in excess bromine and in the presence of a bromination catalyst, usually AlCl₃. The operation is typically conducted at 177° F. (ca. 80.5° C.). The powdered products are not 100% DBDPO, but rather are mixtures that contain up to about 98% DBDPO and about 1.5%, or a little more, of nonabromodiphenyl oxide co-product. As a partially brominated product, this amount of nonabromodiphenyl oxide is considered problematic by some environmental entities.

It would therefore be desirable to provide process technology enabling preparation of DBDPO products of higher purity, such as products comprising (i) at least 99% of DBDPO and (ii) nonabromodiphenyl oxide in an amount not exceeding 0.5%, preferably not exceeding 0.3%, and still more preferably, not exceeding about 0.1%. It would be especially desirable if such technology could produce DBDPO products comprising (i) at least 99.5% of DBDPO and (ii) nonabromodiphenyl oxide in an amount not exceeding 0.5%, preferably not exceeding 0.3%, and still more preferably, not exceeding about 0.1%.

### SUMMARY OF INVENTION

It has now been found possible to directly produce decabromodiphenyl oxide (DBDPO) products having such higher amounts of DBDPO and lower contents of nonabromodiphenyl oxides without recourse to recrystallization or chromatographic purification steps. A feature of this invention is that a fractionation column is not needed to separate HBr from refluxing bromine during the bromination.

An embodiment of this invention is a process for producing decabromodiphenyl oxide from a liquid mixture. The liquid mixture is derived from bromine, a Lewis acid catalyst, and a diphenyl oxide species selected from the group consisting of

(i) diphenyl oxide,
(ii) partially brominated diphenyl oxide,
(iii) decabromodiphenyl oxide having about 0.5% or more nonabromodiphenyl oxide, and
(iv) any combination of (i)-(iii).

### The process comprises distilling bromine and hydrogen bromide from the liquid mixture while feeding bromine to the liquid mixture.

These and other embodiments and features of this invention will be still further apparent from the ensuing description and appended claims.

### FURTHER DETAILED DESCRIPTION OF THE INVENTION

On the basis of studies conducted in our laboratories, one of the prime difficulties in producing high purity decabromodiphenyl oxide (DBDPO) is the existence of an equilibrium between nonabromodiphenyl oxide and decabromodiphenyl oxide. This equilibrium can be depicted as follows:

Br₉−DPO+Br₂ [symbol] Br₁₀−DPO+HBr

Reducing hydrogen bromide content in the reactor enables a shift to the right in this equilibrium so that the amount of nonabromodiphenyl oxide is diminished and more of the desired decabromodiphenyl oxide forms and precipitates with less nonabromodiphenyl oxide being coprecipitated within the decabromodiphenyl oxide particles. Pursuant to this invention, the distillation of bromine and hydrogen bromide from the liquid mixture while feeding bromine to the liquid mixture is deemed to avoid these difficulties.

As used throughout this document, the term “reaction-derived” means that the composition of the product is reaction determined and not the result of use of downstream purification techniques, such as recrystallization or chromatography, or like procedures that can affect the chemical composition of the product. Simple washing steps such as adding water or an aqueous base such as sodium hydroxide to the reaction mixture to inactivate the catalyst and wash away non-chemically bound impurities are not excluded by the term “reaction-derived.” In other words, the products of such high purity are directly produced in the synthesis process apart from use of subsequent purification procedures (other than simple washing steps) as applied to the recovered or isolated products.

For the purposes of this invention, unless otherwise indicated, the % values given for DBDPO and nonabromodiphenyl oxide are to be understood as being the area % values that are derived from gas chromatography analysis. A recommended procedure for conducting such analyses is presented hereinafter. Gas chromatography is a preferred procedure for determining the composition of the products of the processes of this invention.

Since a bromination in excess refluxing bromine is conducted when the diphenyl oxide species is diphenyl oxide and/or partially brominated diphenyl oxide, it is a relatively simple matter to change the conditions slightly to distill bromine and HBr from the liquid mixture. Bromination of diphenyl oxide and/or partially brominated diphenyl oxide is known in the art. See in this connection U.S. Pat. No. 4,778,933.

This invention enables the preparation of highly pure DBDPO products that are derived from diphenyl oxide, partially brominated diphenyl oxide, decabromodiphenyl oxide having about 0.5% or more nonabromodiphenyl oxide, or any combination thereof. Such highly pure DBDPO products can be said to be “reaction-derived” since they are reaction determined and not the result of use of downstream purification techniques, such as recrystallization, chromatography, or like procedures. In other words, products of such high purity are directly produced in the synthesis process apart from use of subsequent purification procedures as applied to the recovered or isolated products. When decabromodiphenyl oxide having about 0.5% or more nonabromodiphenyl oxide is used pursuant to this invention, the processes of this invention can be viewed as a purification process.

The liquid mixture is normally a liquid phase, with a small amount of solids formation as nonabromodiphenyl oxide and/or decabromodiphenyl oxide precipitate. In the practice of this invention, agitation of the liquid mixture is advantageous.

The processes of this invention comprise distilling bromine and hydrogen bromide from the liquid mixture while feeding bromine to the liquid mixture. This means that, rather than separating HBr from bromine, at least some of the HBr-containing bromine is removed from the liquid mixture, while bromine which does not contain HBr is fed into the liquid mixture. That the distillation of the HBr and bromine occurs while the bromine is being fed to the liquid mixture means that there is overlap in their occurrence. The distillation and feed do not need to start at exactly the same moment in time, nor do they need to stop at exactly the same moment in time. Interruptions in the distillation of HBr and bromine, in the feed of bromine, or both, are permissible in the practice of this invention.

It is recommended and preferred that the distillation of bromine and HBr and the feed of bromine commence at or after decabromodiphenyl oxide formation begins. While it is possible to commence the distillation of bromine and HBr and the feed of bromine earlier in the process, no particular advantage is gained by doing so. The point at which decabromodiphenyl oxide formation has begun can be determined analytically by gas chromatography. For liquid mixtures derived from decabromodiphenyl oxide or combinations including decabromodiphenyl oxide, the distillation of bromine and HBr and the feed of bromine can begin upon formation of the liquid mixture. Of course, the distillation of bromine and HBr and the feed of bromine need not begin at the very instant formation of decabromodiphenyl oxide begins; some delay in the initiation of the distillation and/or the feed is acceptable. In some instances, particularly where mixtures involving partially brominated diphenyl oxides and/or decabromodiphenyl oxide are used, the distillation of HBr and bromine and/or the feed of bromine may be commenced before the feed of the diphenyl oxide species to the liquid mixture is completed.

In the practice of this invention, bromine can be fed to the liquid mixture in the liquid state or in the vapor state. Liquid bromine may be fed to the liquid mixture above the surface, at the surface, or below the surface of the liquid mixture. It is preferred to feed liquid bromine subsurface to the liquid mixture. Subsurface feeding of the bromine minimizes the possibility of some of the bromine being fed from being driven off or entrained in the distillation of HBr and bromine. When the bromine is fed as a vapor, it is normally and preferably fed subsurface to the liquid mixture. It is to be noted that when the term “subsurface” is used anywhere in this document, including the claims, the term does not denote that there must be a headspace above the liquid mixture. For example, if the liquid mixture completely fills a reactor (with equal rates of incoming and outgoing flows), the term “subsurface” means in this case that the substance being fed subsurface is being fed directly into the body of the liquid mixture, the surface thereof being defined by the enclosing walls of the reactor.

The distillation and feed may be conducted at atmospheric, subatmospheric, or superatmospheric pressure. The temperature required to effect the distillation of HBr and bromine will vary with the pressure and with the concentrations of HBr and brominated and unbrominated diphenyl oxide species present in the liquid mixture.

One consideration in the operation of the processes of this invention is the moderately low solubility of nonabromodiphenyl oxide and decabromodiphenyl oxide in bromine. Thus, it is desirable to keep enough bromine in the liquid mixture to prevent an acceleration of the precipitation of nonabromodiphenyl oxide and/or decabromodiphenyl oxide, either by adjusting the rate of distillation, the rate of bromine feed, or both rates. In particularly preferred embodiments, the rates are adjusted so that the volume of the liquid mixture is constant or substantially constant.

Excess bromine is used in the Lewis acid catalyzed bromination reaction. The amount of bromine present in the liquid mixture is at least sufficient to maintain a stoichiometric excess relative to the amount of bromine needed to perbrominate the diphenyl oxide and/or partially brominated diphenyl oxide in the liquid mixture. Preferably, the amount of excess bromine in the reaction zone is in the range of about 50 to about 150 mole percent more than the amount theoretically required to perbrominate the feed of diphenyl oxide and/or partially brominated diphenyl oxide. When the diphenyl oxide species is decabromodiphenyl oxide having about 0.5% or more nonabromodiphenyl oxide, the amount of bromine in the liquid mixture is desirably in the range of about 50 to about 150 mole percent more than the amount theoretically required to perbrominate a corresponding amount of diphenyl oxide. Similarly, the amount of bromine fed into the liquid mixture is an amount that substantially continuously maintains such an excess of bromine in the liquid mixture.

Another feature of this invention is that, once separated from the liquid mixture, the HBr in the distillate may be oxidized to form bromine, for example by air oxidation or treatment with hydrogen peroxide. In preferred embodiments, the bromine thus formed can be, and preferably is, recycled to become at least a portion of the bromine being fed to the liquid mixture. Alternatively, the HBr may be separated from the bromine and used or sold. If the HBr is separated from the bromine, the bromine may be used as at least a portion of the bromine being fed to the liquid mixture.

Termination of the bromination reaction is typically effected by deactivating the catalyst with water and/or an aqueous base such as a solution of sodium hydroxide or potassium hydroxide.

The Lewis acid catalyst in the liquid mixture can be any of various iron and/or aluminum Lewis acids. These include the metals themselves such as iron powder, aluminum foil, or aluminum powder, or mixtures thereof. Preferably use is made of such catalyst materials as, for example, ferric chloride, ferric bromide, aluminum chloride, aluminum bromide, or mixtures of two or more such materials. More preferred are aluminum chloride and aluminum bromide with addition of aluminum chloride being more preferred from an economic standpoint. It is possible that the makeup of the catalyst may change when contained in the liquid mixture. The Lewis acid should be employed in an amount sufficient to effect a catalytic effect upon the bromination reaction being conducted. Typically, the amount of Lewis acid used will be in the range of about 0.06 to about 2 wt %, and preferably in the range of about 0.2 to about 0.7 wt % based on the weight of the bromine being used.

In the various embodiments of this invention, the diphenyl oxide species can be diphenyl oxide (DPO) itself, one or a mixture of partially brominated diphenyl oxides, decabromodiphenyl oxide having about 0.5% or more nonabromodiphenyl oxide, a mixture of DPO and one or more partially brominated diphenyl oxides, a mixture of DPO and decabromodiphenyl oxide having about 0.5% or more nonabromodiphenyl oxide, a mixture of one or more partially brominated diphenyl oxides and decabromodiphenyl oxide having about 0.5% or more nonabromodiphenyl oxide, or a mixture of DPO, one or more partially brominated diphenyl oxides, and decabromodiphenyl oxide having about 0.5% or more nonabromodiphenyl oxide.

Partially brominated DPO, which can be used in the practice of this invention, typically contains on average in the range of about 0.5 to about 6 atom(s) of bromine per molecule, preferably in the range of about 2 to about 4 bromine atoms per molecule. Partially brominated diphenyl oxides with more than about 6 atoms of bromine per molecule can be used in the processes of this invention. The processes of this invention can be applied to any decabromodiphenyl oxide, but are especially useful for decabromodiphenyl oxide that contains about 0.5% or more nonabromodiphenyl oxide.

The DBDPO products formed in processes of this invention are white or slightly off-white in color. White color is advantageous as it simplifies the end-user's task of insuring consistency of color in the articles that are flame retarded with the DBDPO products.

The DBDPO products formed in the processes of this invention may be used as flame retardants in formulations with virtually any flammable material. The material may be macromolecular, for example, a cellulosic material or a polymer. Illustrative polymers are: olefin polymers, cross-linked and otherwise, for example homopolymers of ethylene, propylene, and butylene; copolymers of two or more of such alkene monomers and copolymers of one or more of such alkene monomers and other copolymerizable monomers, for example, ethylene/propylene copolymers, ethylene/ethyl acrylate copolymers and ethylene/propylene copolymers, ethylene/acrylate copolymers and ethylene/vinyl acetate copolymers; polymers of olefinically unsaturated monomers, for example, polystyrene, e.g. high impact polystyrene, and styrene copolymers, polyurethanes; polyamides; polyimides; polycarbonates; polyethers; acrylic resins; polyesters, especially poly(ethyleneterephthalate) and poly(butyleneterephthalate); polyvinyl chloride; thermosets, for example, epoxy resins; elastomers, for example, butadiene/styrene copolymers and butadiene/acrylonitrile copolymers; terpolymers of acrylonitrile, butadiene and styrene; natural rubber; butyl rubber and polysiloxanes. The polymer may be, where appropriate, cross-linked by chemical means or by irradiation. The DBDPO products of this invention can be used in textile applications, such as in latex-based back coatings.

The amount of a DBDPO product of this invention used in a formulation will be that quantity needed to obtain the flame retardancy sought. It will be apparent to those skilled in the art that for all cases no single precise value for the proportion of the product in the formulation can be given, since this proportion will vary with the particular flammable material, the presence of other additives and the degree of flame retardancy sought in any given application. Further, the proportion necessary to achieve a given flame retardancy in a particular formulation will depend upon the shape of the article into which the formulation is to be made, for example, electrical insulation, tubing, electronic cabinets and film will each behave differently. In general, however, the formulation, and resultant product, may contain from about 1 to about 30 wt %, preferably from about 5 to about 25 wt % DBDPO product of this invention. Masterbatches of polymer containing DBDPO, which are blended with additional amounts of substrate polymer, typically contain even higher concentrations of DBDPO, e.g., up to 50 wt % or more.

It is advantageous to use the DBDPO products of this invention in combination with antimony-based synergists, e.g., Sb₂O₃. Such use is conventionally practiced in all DBDPO applications. Generally, the DBDPO products of this invention will be used with the antimony based synergists in a weight ratio ranging from about 1:1 to 7:1, and preferably of from about 2:1 to about 4:1.

Any of several conventional additives used in thermoplastic formulations may be used, in their respective conventional amounts, with the DBDPO products of this invention, e.g., plasticizers, antioxidants, fillers, pigments, UV stabilizers, etc.

Thermoplastic articles formed from formulations containing a thermoplastic polymer and DBDPO product of this invention can be produced conventionally, e.g., by injection molding, extrusion molding, compression molding, and the like. Blow molding may also be appropriate in certain cases.

### Recommended Gas Chromatographic Procedure

The gas chromatography is on a Hewlett-Packard 5890 gas chromatograph using a 12QC5 HTS capillarycolumn, 12 meter, 0.15μ film thickness, 0.53 mm diameter, part number 054657, available from SGE, Inc, (SGE Inc., 2007 Kramer Lane, Austin, Tex. 78758). Conditions were: 1:10 split injection, column head pressure 9 psig (ca. 1.63×10⁵ Pa), injector temperature 325° C., flame ionization detector temperature 350° C., and column temperature 300° C. isothermal. The carrier gas was helium. Samples were prepared by dissolving, with warming, 0.05 grams in 10 mL of dibromomethane and injection of 1 microliter of this solution. The integration of the peaks was carried out using Target Chromatography Analysis Software from Thru-Put Systems, Inc. (5750 Major Blvd., Suite 200, Orlando Fla. 32819; currently owned by Thermo Lab Systems). However, other and commercially available software suitable for use in integrating the peaks of a chromatograph may be used.

The GC procedure described above provides a trace having several peaks. The first peak is deemed to be the meta- and para-hydrogen isomers of nonabromodiphenyl oxide. The second peak is deemed to be the ortho-hydrogen isomer of nonabromodiphenyl oxide. The main peak, of course, is decabromodiphenyl oxide.

The following example is presented for purposes of illustration, and is not intended to impose limitations on the scope of this invention.

### EXAMPLE 1

A reactor is configured from a 1-liter Morton flask with a mechanical stirrer, thermometer, a 60 mL addition funnel, and a distillation column. The condenser from the distillation column is connected to a H₂O trap. A small N₂ purge is added to the line from the condenser to the H₂O trap. The reactor is charged with AlCl₃ and bromine. The addition funnel is charged with diphenyl oxide. The reactor is heated to 55° C. and the diphenyl oxide is added drop-wise supersurface to the bromine. The reactor is heated by a mantle. After all of the diphenyl oxide has been added, the addition funnel is replaced with a Br₂ feed line. After several minutes of refluxing, the distillation of Br₂ (containing HBr) is initiated. At the same time, the Br₂ feed is initiated. As needed, the feed rate of the Br₂ is adjusted so that the volume in the reactor remains fairly constant. After the distillation and concurrent replacement feed of Br₂ are conducted for an hour, the liquid mixture is cooled to 55° C., some deionized H₂O is added, and most of the Br₂ is distilled off. When most of the Br₂ is gone, more deionized water is added. The remaining Br₂ is then distilled. The remaining mixture is cooled to 60° C., and a portion of an aqueous 25% NaOH solution is added to make the pH 13-14. The resultant mixture is filtered and washed well with deionized water. A sample is subjected to GC analysis and then is oven dried.

It is to be understood that the reactants and components referred to by chemical name or formula anywhere in this document, whether referred to in the singular or plural, are identified as they exist prior to coming into contact with another substance referred to by chemical name or chemical type (e.g., another reactant, a solvent, or etc.). It matters not what preliminary chemical changes, transformations and/or reactions, if any, take place in the resulting mixture or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical operation or reaction or in forming a mixture to be used in conducting a desired operation or reaction. Also, even though an embodiment may refer to substances, components and/or ingredients in the present tense (“is comprised of”, “comprises”, “is”, etc.), the reference is to the substance, component or ingredient as it existed at the time just before it was first contacted, blended or mixed with one or more other substances, components and/or ingredients in accordance with the present disclosure.

Also, even though the claims may refer to substances in the present tense (e.g., “comprises”, “is”, etc.), the reference is to the substance as it exists at the time just before it is first contacted, blended or mixed with one or more other substances in accordance with the present disclosure.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

Each and every patent or other publication or published document referred to in any portion of this specification is incorporated in toto into this disclosure by reference, as if fully set forth herein.

This invention is susceptible to considerable variation within the spirit and scope of the appended claims.

1. A process for producing decabromodiphenyl oxide from a liquid mixture, wherein said liquid mixture is derived from bromine, a Lewis acid catalyst, and a diphenyl oxide species selected from the group consisting of
(i) diphenyl oxide,
(ii) partially brominated diphenyl oxide,
(iii) decabromodiphenyl oxide having about 0.5% or more nonabromodiphenyl oxide, and
(iv) any combination of (i)-(iii),
which process comprises distilling bromine and hydrogen bromide from the liquid mixture while feeding bromine to the liquid mixture.

2. A process as in claim 1 wherein the bromine being fed to the liquid mixture is in the liquid state.

3. A process as in claim 1 wherein the bromine being fed to the liquid mixture is in the vapor state.

4. A process as in claim 1 wherein, after said distilling, the hydrogen bromide is oxidized to form bromine.

5. A process as in claim 4 wherein the bromine formed by oxidizing hydrogen bromide is recycled as at least a portion of the bromine being fed to the liquid mixture.

6. A process as in claim 1 wherein, after said distilling, the hydrogen bromide is separated from the bromine.

7. A process as in claim 6 wherein the bromine separated from the hydrogen bromide is recycled as at least a portion of the bromine being fed to the liquid mixture.

8. A process as in claim 1 wherein said feeding is subsurface to the liquid mixture.

9. A process as in claims 1 wherein said process is conducted at atmospheric pressure.

10. A process as in claim 1 wherein said diphenyl oxide species is (i) diphenyl oxide.

11. A process as in claim 1 wherein said diphenyl oxide species is (ii) partially brominated diphenyl oxide.
